(19) United States
(12) Patent Application Publication
(10) Pub. No.: US 2006/0269507 A1
(43) Pub. Date: Nov. 30, 2006

(54) Topical ointment compostion and method for making the same

(76) Inventor: Susan Fuller, Orlando, FL (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/140,234

(22) Filed: May 27, 2005

(51) Int. Cl. A61K 36/889; A61K 8/97; A61K 36/53; A61K 36/898

(52) U.S. Cl. 424/74; 424/727; 424/745; 424/776

(57) ABSTRACT

A topical ointment composition comprises a mixture of organic beeswax, organic cocoa butter, virgin coconut oil, organic shea butter, cold-pressed extra virgin olive oil, pure lavender essential oil and benzoin tincture.

### BACKGROUND OF THE INVENTION

This invention relates generally to topical ointments, creams or lotions. More particularly, this invention pertains to such ointments or creams that comprise oils and waxes that are minimally processed and do not contain artificial or synthetic ingredients or components.

Millions of people suffer from temporary or chronic skin problems including dry skin, psoriasis, eczema, bruising, rashes, allergic reactions, minor burns and the like. A variety of topical skin-care products are available on the market. However, many of these products employ synthetic compounds such as ammonia, acids, alcohol's, animal oils, artificial color, cobalt, mineral oil, silicones, lead nitrates, iron salts, oxides, and petroleum derivatives that may cause skin irritation, and/or allergic reactions. Such products may cause allergic reactions when applied to the skin only compounding the problem.

In addition, products are available that utilize organic ingredients without artificial compounds. However such products may combine an extensive list of ingredients that unnecessarily complicate the manufacturing process and increase the cost of the product or dilute the effective ingredients. Accordingly, a need exists for a topical ointment that treats a variety of skin care problems; incorporates organic ingredients at concentrated levels to enhance the potent effectiveness of the components without compromising the integrity of the composition; provides satisfactory skin penetration and supplying properties to leave skin smooth for an extended period of time, provides organic moisturizing compositions that provide a water resistant antiseptic film on the skin; provide a high volume of organic cold pressed olive oil for immediate transdermal delivery and has other general healing properties, that are inexpensive and simple to manufacture.

### SUMMARY OF THE INVENTION

The present invention for a topical ointment composition comprises a mixture of organic waxes, oils and/ butters, one or more of which may have anti-fungal, anti-bacterial, anti-microbial or preservative properties; as well as general skin care properties such as antiseptic, moisturizing, lubricating, and hydrating. The composition comprises a mixture of organic beeswax, organic cocoa butter, virgin coconut oil, organic shea butter and cold-pressed extra virgin olive oil. Lavender oil may be added for fragrance and its anti-inflammatory properties. Benzoin tincture may be added as a natural preservative. This composition has been found to alleviate a variety of skin conditions for example: chronic chapped or dry skin, eczema, heat rashes, minor burns, stretch marks, diaper rash, wrinkles and reducing skin discoloration. The composition has been found to be useful to treat such skin conditions on humans and animals.

### DESCRIPTION OF PREFERRED EMBODIMENT

The present invention for a topical ointment comprises a mixture of waxes, oils, butters and an organic preservative to control bacterial growth. More specifically the mixture comprises beeswax, cocoa butter, virgin coconut oil, shea butter, cold-pressed extra virgin olive oil, a pure lavender essential oil and benzoin tincture. These ingredients are organic in the sense the ingredients are minimally processed and not manufactured with the employment synthetic ingredients or methods. The mixture preferably contains from about 7% to about 17% by weight of beeswax, from about 21% to about 29% by weight of cocoa butter, from about 21% to about 29% by weight of virgin coconut oil, from about 21% to about 29% by weight of shea butter, from about 11.5% to about 21.5% by weight of the extra virgin olive oil. In addition, from about 0.1 to 20 milliliters of the lavender oil and benzoin tincture may be added to the composition.

For purposes of describing the invention, specific amounts of the ingredients are provided, but the invention is not so limited. Varying amounts may be used as long as the above-described percentages or range of percentages, or range of volumetric amounts, are maintained in the making the composition. The below described process or method can be conducted at one's home in the kitchen, but the invention can be made on a larger scale.

An exemplary embodiment of the invention comprises the following ingredients:
4 ounces (weight measurement) of organic beeswax
8 ounces (weight measurement) of organic cocoa butter
8 ounces (weight measurement) of virgin coconut oil
8 ounces (weight measurement) of organic shea butter
8 ounces (volume measurement which is equal to about 5.5 ounces by weight) of cold-pressed extra virgin olive oil;
0.1 to 20 milliliters of lavender essential oil; and
0.1 to 20 milliliters of benzoin tincture.

The above listed ingredients are preferably mixed in a glass container under heat. The container is heated from about 220° F. to about 230° Fahrenheit. In an exemplary embodiment, the beeswax and the cocoa butter are melted to a liquid in the glass container. The beeswax and cocoa butter are preferably shredded prior to placement in the container to expedite the melting of these ingredients. The beeswax and cocoa butter may be purchased from various suppliers, for example Tropical Blossom Honey Company located in Edgewater, Fla. sells beeswax, and Let Sleeping Dogs Lye Soaps, located in Halstead, Kans. sells cocoa butter.

After the beeswax and cocoa butter are melted to a liquid, the virgin coconut oil and organic shea butter are added. Each of these products is solid but become liquid at approximately 76° Fahrenheit, and quickly melt upon addition to the mixture. The beeswax and shea butter provide essential polyunsaturated oil lipids the skin needs to maintain its healthy condition. A supplier of virgin coconut oil is Tropical Traditions, Inc. located in Mabel, Minn. A supplier of the shea butter is Shea Terra Organics located in Fairfax, Va.

Once the coconut oil and shea butter have melted, the heat source is terminated. Then eight (8) ounces (volumetric measurement) of virgin olive oil is added, and the mixture is stirred. As it cools it thickens to form a cream. The mixture should have the consistency of pudding once it is cooled. The virgin olive oil is understood to provide a swift transdermal delivery and penetration of the composition to the dermis. Extra virgin olive oil may be purchased from Whole Foods Market in Austin, Tex.

The composition is allowed to cool to room temperature, at which point about about 0.1 milliliters to about 20 milliliters of lavender essential oil and benzoin tincture are added and the composition is stirred a sufficient amount of time to adequately mix these ingredients in the composition. After mixing the composition may be placed in a jar in which the composition continues to set up to a consistency of a vegetable shortening.

It has been found that by the use of these effective ingredients at higher concentrations than found in other skin-care products, it eliminates the need for additional preservatives that may dilute the concentrations of the effective ingredients. The shea butter and coconut oil, for example, have anti-bacterial and anti-fungal properties. When used at these disclosed concentrations, and in combination with the benzoin tincture, (which also has preservative characteristics), no additional compounds or ingredients are needed to act as a preservative. Moreover, the increased concentration levels promote the lubricating, moisturizing and hydrating characteristics of the shea butter and coconut oil. The beeswax bonds the lubricating, moisturizing, antispectic and hydrating compounds to the skin for up to twelve hours.

In use, the composition is applied to and an affected area of the skin. This formulation has a low melting point, thereby eliminating and preventing skin drag on sensitive and irritated skin. Depending on the nature of the skin infirmity, the composition may be applied one or more times a day. The improved composition has been topically tested on various skin problems as set forth in the foregoing examples.

### EXAMPLE 1

The composition was applied once a day at night before bedtime to itching and inflamed eczema on the arm of a ten (10) year old girl. Overnight the redness was significantly reduced. Within only one week, the eczema had almost entirely dissipated.

### EXAMPLE 2

A pregnant woman who had stretch marks on and around her torso from a previous pregnancy applied the ointment composition once a day through the term of her second pregnancy. The stretch marks disappeared and the woman did not obtain additional stretch marks from the second pregnancy, despite gaining eighty-five pounds.

### EXAMPLE 3

An adult male obtained a chemical burn on his hand causing his finger to become inflamed and slightly blistered. Other skin care products were used but were not effective in reducing the inflammation. The composition was applied once a day for three to four days, and eliminated the inflammation and blistering.

### EXAMPLE 4

A ten year old boy often suffered from heat rash in the groin area as a result of the playing softball in the summer heat. The composition was applied once before the boy went to bed. The following morning the inflammation of the rash was only slightly pink, and the boy did not experience any irritation at areas of the rash.

While the preferred embodiments of the present invention have been shown and described herein, it will be obvious that such embodiments are provided by way of example only and not of limitation. Numerous variations, changes and substitutions will occur to those skilled in the art without departing from the teaching of the present invention. Accordingly, it is intended that the invention be interpreted within the full spirit and scope of the appended claims.

What I claim as my invention is:

1. A topical ointment composition, comprising: an effective amount of beeswax, cocoa butter, virgin coconut oil, shea butter, virgin olive oil, lavender oil and benzoin tincture.

2. A topical ointment composition, comprising:
about 7% to about 17% by weight beeswax;
about 21% to about 29% weight of cocoa butter;
about 21% to about 29% by weight of virgin coconut oil;
about 21% to about 29% by weight of shea butter; and,
about 11.5% to about 21.5% by weight of the extra virgin olive oil_{[cs1]}.

3. The composition of claim 2 further comprising about 0.1 milliliters to about 20 milliliters of lavender oil and about 0.1 milliliters to about 20 milliliters of benzoin tincture.

4. A topical ointment composition, comprising:
4 ounces by weight of organic beeswax;
8 ounces by weight of organic cocoa butter;
8 ounces by volume of virgin coconut oil;
8 ounces by weight of organic shea butter;
0.1 milliliters to 20 milliliters of lavender oil; and,
0.1 milliliters to 20 milliliters of benzoin tincture.

5. A method of preparing a topical ointment composition, comprising the steps of:
providing a container in which the composition is made;
heating the container to within a predetermined temperature range;
placing predetermined amounts of beeswax, cocoa butter, and shea butter in the container and melting within the container predetermined amounts of beeswax, cocoa butter, and shea butter;
adding virgin coconut oil while the container is heated within the predetermined temperature range;
removing the heat source from the container;
adding virgin-olive to the composition before the composition cools to room temperature; and,
adding lavender oil and benzoin tincture to the composition after the composition cools to room temperature.

6. The method of claim 5 further comprising the step of shredding the beeswax and cocoa butter prior to placing the beeswax and cocoa butter in the container.
